(19) United States
(12) Patent Application Publication
(10) Pub. No.: US 2016/0298110 A1
(43) Pub. Date: Oct. 13, 2016

(54) MODIFIED SURFACES

(71) Applicants: Glenn MCGALL, Palo Alto, CA (US); CENTRILLION TECHNOLOGY HOLDINGS CORPORATION, Cayman Islands (KY)

(72) Inventor: Glenn McGall, Palo Alto, CA (US)

(21) Appl. No.: 15/101,168

(22) PCT Filed: Dec. 5, 2014
(86) PCT No.: PCT/US14/68947

§ 371 Date: Jun. 2, 2016

Related U.S. Application Data
(60) Provisional application No. 61/912,027, filed on Dec. 5, 2013.
(60) Provisional application No. 61/979,431, filed on Apr. 14, 2014.

(51) Int. Cl. C12N 15/10; C08F 220/58; C12Q 1/68

(52) U.S. Cl. CPC C12N 15/1093; C12Q 1/6874; C12Q 1/686; C08F 220/58

(57) ABSTRACT

Provided herein are methods and compositions for coating surfaces with polymers. The methods and compositions are suited for conducting biological reactions.

### CROSS-REFERENCE

This application claims the benefit of U.S. Provisional Application No. 61/912,027, filed Dec. 5, 2013, and U.S. Provisional Application No. 61/979,431, filed Apr. 14, 2014, which applications are incorporated herein by reference.

### BACKGROUND

In many sequencing by synthesis (SBS) systems, clonal amplification and SBS are performed in glass flow cell channels. PCR primers are attached to the inner surface of the channels via a passively bound polymer coating. Weakly bound polymer chains are washed away prior to use, but the remaining polymer can become depleted to varying extents during extensive cycles of SBS, causing progressive loss of signal. This is a particular concern when high pH and elevated temperature conditions are employed.

### SUMMARY

Methods and compositions are provided for fabricating polymer coatings by surface initiated polymerization incorporating biomolecules. In some cases, the compositions and methods are useful in performing nucleic acid reactions and sequencing by synthesis. In some cases, the compositions and methods are useful in providing coatings that are robust.

An aspect of the present disclosure provides a composition, comprising: a surface with a 10 or more nucleic acid molecules coupled thereto, wherein at least 90% of the nucleic acid molecules remain intact and coupled to the surface after at least 30 PCR cycles, wherein each PCR cycle comprises the following reaction conditions: (a) a denaturation step at a temperature of at least 85° C. for at least 15 seconds; (b) an annealing step at a temperature of at least 50° C. for at least 15 seconds; and (c) an extension step at a temperature of at least 70° C. for at least 30 seconds.

In some embodiments of aspects provided herein, the surface is covered with a polymer brush. In some embodiments of aspects provided herein, the polymer brush comprises acrylamide. In some embodiments of aspects provided herein, the polymer brush further comprises N-(2-hydroxyethyl)acrylamide. In some embodiments of aspects provided herein, at least 1,000 different nucleic acid molecules are coupled to the surface. In some embodiments of aspects provided herein, at least 100,000 different nucleic acid molecules are coupled to the surface. In some embodiments of aspects provided herein, at least 1,000,000 different nucleic acid molecules are coupled to the surface.

An aspect of the present disclosure provides a method for performing an enzymatic reaction, comprising: (a) providing a substrate having a polymer brush coating and a plurality of biomolecules coupled to the polymer brush; and (b) performing one or more enzymatic reactions with the biomolecules on the substrate.

In some embodiments of aspects provided herein, the biomolecules are selected from the group consisting of: oligonucleotides, polynucleotides, aptamers, proteins, and antibodies. In some embodiments of aspects provided herein, the enzymatic reaction is selected from the group consisting of: polymerase chain reaction, sequencing reaction, ligation reaction, extension reaction, and transcription reaction. In some embodiments of aspects provided herein, further comprises applying heat to the substrate. In some embodiments of aspects provided herein, at least 90% of the biomolecules are retained with at least 90% integrity after 40 cycles of sequencing by synthesis reactions. In some embodiments of aspects provided herein, at least 90% of the biomolecules are retained with at least 90% integrity after 25 cycles of polymerase chain reactions. In some embodiments of aspects provided herein, the substrate comprises at least 1,000,000 different types of biomolecules, and wherein each biomolecule is an oligonucleotide. In some embodiments of aspects provided herein, the enzymatic reaction is an extension reaction.

An aspect of the present disclosure provides a method for making a modified surface, comprising: (a) providing a surface; (b) covalently bonding initiator species to the surface; (c) conducting surface initiated polymerization of a polymer from the initiator species, thereby producing a polymer coating comprising a plurality of polymer chains; and (d) coupling two or more different biomolecules to the polymer coating.

An aspect of the present disclosure provides a method for making a modified surface, comprising: (a) providing a surface; (b) covalently bonding initiator species to the surface; (c) conducting surface initiated polymerization of a mixture two or more different types of acrylamide monomers from the initiator species, thereby producing a polymer coating comprising a plurality of polymer chains; and (d) coupling biomolecules to the polymer coating.

In some embodiments of aspects provided herein, the biomolecules are selected from the group consisting of: oligonucleotides, polynucleotides, aptamers, proteins, and antibodies. In some embodiments of aspects provided herein, the two or more different biomolecules are two different oligonucleotides. In some embodiments of aspects provided herein, the two or more different types of acrylamide monomers are selected from the group consisting of: acrylamide, N-(2-hydroxyethyl)acrylamide, ethylene glycol acrylamide, and hydroxyethylmethacrylate (HEMA). In some embodiments of aspects provided herein, the surface is selected from the group consisting of glass, silica, titanium oxide, aluminum oxide, indium tin oxide (ITO), silicon, polydimethylsiloxane (PDMS), polystyrene, polycyclicolefins, polymethylmethacrylate (PMMA), titanium, and gold. In some embodiments of aspects provided herein, the surface comprises glass. In some embodiments of aspects provided herein, the surface comprises silicon. In some embodiments of aspects provided herein, the surface is selected from the group consisting of: flow cells, sequencing flow cells, flow channels, microfluidic channels, capillary tubes, piezoelectric surfaces, wells, microwells, microwell arrays, microarrays, chips, wafers, non-magnetic beads, magnetic beads, ferromagnetic beads, paramagnetic beads, superparamagnetic beads, and polymer gels. In some embodiments of aspects provided herein, the initiator species comprises an organosilane. In some embodiments of aspects provided herein, the initiator species comprises the molecule shown in FIG. 1. In some embodiments of aspects provided herein, the surface initiated polymerization comprises atom-transfer radical polymerization (ATRP). In some embodiments of aspects provided herein, the surface initiated polymerization comprises reversible addition fragmentation chain-transfer (RAFT). In some embodiments of aspects provided herein, the biomolecules comprise 5′ acrydite modified oligonucleotides. In some embodiments of aspects provided herein, the biomolecules comprise antibodies. In some embodiments of aspects provided herein, the biomolecules comprise peptides. In some embodiments of aspects provided herein, the biomolecules comprise aptamers. In some embodiments of aspects provided herein, the coupling of the biomolecules comprises incorporation of acrydite-modified biomolecules during polymerization. In some embodiments of aspects provided herein, the biomolecules comprises reaction at bromoacetyl sites. In some embodiments of aspects provided herein, the coupling of the biomolecules comprises reaction at azide sites. In some embodiments of aspects provided herein, the coupling of the biomolecules comprises azide-alkyne Huisgen cycloaddition.

An aspect of the present disclosure provides a composition, comprising: (a) a surface; (b) a polymer coating covalently bound to the surface, formed by surface-initiated polymerization, wherein the polymer coating comprises 2 or more different types of acrylamide monomers; and (c) a biomolecule coupled to the polymer coating.

An aspect of the present disclosure provides a composition, comprising: (a) a surface; (b) a polymer coating covalently bound to the surface, formed by surface-initiated polymerization; and (c) at least two different biomolecules coupled to the polymer coating.

In some embodiments of aspects provided herein, the biomolecule comprises an oligonucleotide. In some embodiments of aspects provided herein, the oligonucleotide is coupled to the polymer at its 5′ end. In some embodiments of aspects provided herein, the oligonucleotide is coupled to the polymer at its 3′ end. In some embodiments of aspects provided herein, the biomolecule comprises an antibody. In some embodiments of aspects provided herein, the biomolecule comprises an aptamer. In some embodiments of aspects provided herein, the at least two different biomolecules comprise oligonucleotides. In some embodiments of aspects provided herein, the oligonucleotides are coupled to the polymer coating at their 5′ ends. In some embodiments of aspects provided herein, the oligonucleotides are coupled to the polymer coating at their 3′ ends. In some embodiments of aspects provided herein, the at least two different biomolecules comprise antibodies. In some embodiments of aspects provided herein, the at least two different biomolecules comprise aptamers. In some embodiments of aspects provided herein, the surface comprises glass. In some embodiments of aspects provided herein, the surface comprises silicon. In some embodiments of aspects provided herein, the polymer coating comprises polyacrylamide. In some embodiments of aspects provided herein, the polymer coating comprises PMMA. In some embodiments of aspects provided herein, the polymer coating comprises polystyrene. In some embodiments of aspects provided herein, the surface-initiated polymerization comprises atom-transfer radical polymerization (ATRP). In some embodiments of aspects provided herein, the surface-initiated polymerization comprises reversible addition fragmentation chain-transfer (RAFT).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of an initiator silane.

FIG. 2 shows an example of a phosphorylcholine-acrylamide monomer.

FIG. 3 shows an example of a betaine-acrylamide monomer.

FIG. 4 shows an example of a process for producing a polyacrylamide surface coating with oligonucleotides.

### DETAILED DESCRIPTION OF THE INVENTION

Overview

This disclosure provides methods and compositions for improved polymer coatings on surfaces. The polymer coatings can be generated via surface-initiated polymerization (SIP) via initiator species bound to a surface. The polymer coatings can incorporate modified monomers to modulate physicochemical properties of the coatings. The polymer coatings can incorporate oligonucleotides.

Surfaces

The methods and compositions provided in this disclosure can comprise creating a polymer coating on a surface. The surface can comprise glass, silica, titanium oxide, aluminum oxide, indium tin oxide (ITO), silicon, polydimethylsiloxane (PDMS), polystyrene, polyolefins, such as Poly(methylpentene) (PMP) and Zeonor™, cyclic olefin copolymer such as Topas™, polymethylmethacrylate (PMMA), other plastics, titanium, gold, other metals, or other suitable materials. The surface can be flat or round, continuous or non-continuous, smooth or rough. Examples of surfaces include flow cells, sequencing flow cells, flow channels, microfluidic channels, capillary tubes, piezoelectric surfaces, wells, microwells, microwell arrays, microarrays, chips, wafers, non-magnetic beads, magnetic beads, ferromagnetic beads, paramagnetic beads, superparamagnetic beads, and polymer gels.

Initiator Species Attachment

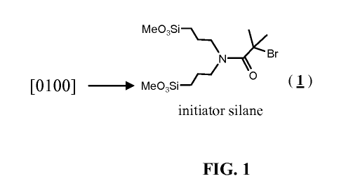

The methods and compositions provided in this disclosure can comprise initiator species for bonding to a support surface. In some cases, the initiator species comprises at least one organosilane. The organosilane can comprise one surface-bonding group, resulting in a mono-pedal structure. The organosilane can comprise two surface-bonding groups, resulting in a bi-pedal structure. The organosilane can comprise three surface-bonding groups, resulting in a tri-pedal structure. The surface bonding group can comprise MeO₃Si (e.g. see FIG. 1, item [0100]). The surface bonding group can comprise (MeO)₃Si. The surface bonding group can comprise (EtO)₃Si. The surface bonding group can comprise (AcO)₃Si. The surface bonding group can comprise (Me₂N)₃Si. The surface bonding group can comprise (HO)₃Si. For cases where the organosilane comprises multiple surface bonding groups, the surface bonding groups can be the same or can be different. The organosilane can comprise the silane reagent shown in FIG. 1. In some cases, the initiator species comprises at least one organophosphonic acid, wherein the surface bonding group comprises (HO)₂P(═O). The organophosphonic acid can comprise one surface-bonding group, resulting in a mono-pedal structure. The organophosphonic acid can comprise two surface-bonding groups, resulting in a bi-pedal structure. The organophosphonic acid can comprise three surface-bonding groups, resulting in a tri-pedal structure.

Silane treatment of substrates (e.g., glass substrates) can be performed with a silane solution, such as a solution of silane in ethanol, water, or a mixture thereof. Prior to treatment with a silane solution, a substrate can be cleaned. Cleaning can be performed by immersion in sulfuric-peroxide solution. For attachment of an initiator species to a plastic substrate, a thin film of silica can be applied to the surface. Silica can be deposited by a variety of methods, such as vacuum deposition methods including but not limited to chemical vapor deposition (CVD), sputtering, and electron-beam evaporation. Silane treatment can then be performed on the deposited silica layer.

Surface-Initiated Polymerization (SIP)

The methods and compositions provided in this disclosure can comprise forming a polymer coating from surface-bound initiator species. The resulting polymer coatings can comprise linear chains. The resulting polymer coatings can comprise lightly branched chains. The polymer coatings can form polymer brush thin-films. The polymer coatings can include some cross-linking The polymer coatings can form a graft structure. The polymer coatings can form a network structure. The polymer coatings can form a branched structure. The polymers can comprise homogenous polymers. The polymers can comprise block copolymers. The polymers can comprise gradient copolymers. The polymers can comprise periodic copolymers. The polymers can comprise statistical copolymers.

Polymer coatings can comprise polymer molecules of a particular length or range of lengths. Polymer molecules can have a length of at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, or 500 backbone atoms or molecules (e.g., carbons). Polymer molecules can have a length of at most 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, or 500 backbone atoms or molecules (e.g., carbons). Polymer molecules can have a length of at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, or 500 monomer units (e.g., acrylamide molecules). Polymer molecules can have a length of at most 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, or 500 monomer units (e.g., acrylamide molecules).

The polymer can comprise polyacrylamide (PA). The polymer can comprise polymethylmethacrylate (PMMA). The polymer can comprise polystyrene (PS). The polymer can comprise polyethylene glycol (PEG). The polymer can comprise polyacrylonitrile (PAN). The polymer can comprise poly(styrene-r-acrylonitrile) (PSAN). The polymer can comprise a single type of polymer. The polymer can comprise multiple types of polymer. The polymer can comprise any of the polymers described in “Ayres, N. (2010). Polymer brushes: Applications in biomaterials and nanotechnology. Polymer Chemistry, 1(6), 769-777,” or in “Barbey, R., Lavanant, L., Paripovic, D., Schüwer, N., Sugnaux, C., Tugulu, S., & Klok, H. A. (2009). Polymer brushes via surface-initiated controlled radical polymerization: synthesis, characterization, properties, and applications. Chemical reviews, 109(11), 5437-5527.”

The polymerization can comprise methods to control polymer chain length, coating uniformity, or other properties. The polymerization can comprise controlled radical polymerization (CRP). The polymerization can comprise atom-transfer radical polymerization (ATRP). The polymerization can comprise reversible addition fragmentation chain-transfer (RAFT). The polymerization can comprise living polymerization processes, including those described in “Ayres, N. (2010). Polymer brushes: Applications in biomaterials and nanotechnology. Polymer Chemistry, 1(6), 769-777,” or in “Barbey, R., Lavanant, L., Paripovic, D., Schüwer, N., Sugnaux, C., Tugulu, S., & Klok, H. A. (2009). Polymer brushes via surface-initiated controlled radical polymerization: synthesis, characterization, properties, and applications. Chemical reviews, 109(11), 5437-5527.”

Incorporation of Biomolecules

Biomolecules can be coupled to the polymer coatings described in this disclosure. The biomolecules can comprise antibodies. The biomolecules can comprise proteins. The biomolecules can comprise peptides. The biomolecules can comprise enzymes. The biomolecules can comprise aptamers. The biomolecules can comprise oligonucleotides.

Oligonucleotides can be coupled to the polymer coatings described in this disclosure. The oligonucleotides can comprise primers. The oligonucleotides can comprise cleavable linkages. Cleavable linkages can be enzymatically cleavable. The oligonucleotides can comprise at least 1, 2, 3, 4, 5, 6, 7, 8, 9, 10, 11, 12, 13, 14, 15, 16, 17, 18, 19, 20, 25, 30, 35, 40, 45, 50, 55, or 60 bases. The oligonucleotides can vary in length, such as from 3 to 5 bases, from 1 to 50 bases, from 6 to 12 bases, from 8 to 12 bases, from 15 to 25 bases, from 25 to 35 bases, from 35 to 45 bases, or from 45 to 55 bases. The individual oligonucleotides coupled to the coatings can differ from each other in length.

Biomolecules (e.g., oligonucleotides) can be incorporated into the polymer coatings during the polymerization process. For example, 5′-acrydite-modified oligonucleotides chains can be added during the polymerization process to allow the incorporation of the oligonucleotides into the polymerizing polyacrylamide structure. In some cases, oligonucleotides are coupled to the polymer coating at the 5′ end. In some cases, oligonucleotides are coupled to the polymer coating at the 3′ end. In some cases, some oligonucleotides are coupled to the polymer coating at the 3′ end and some oligonucleotides are coupled to the polymer coating at the 5′ end.

Biomolecules (e.g., oligonucleotides) can be incorporated into the polymer coatings after the polymerization process. For example, reactive sites can be added to the polymer structure during the polymerization process. Biomolecules can be incorporated at the reactive sites subsequent to the polymerization. The reactive sites can comprise bromoacetyl sites. The reactive sites can comprise azides. The reactive sites can comprise sites compatible with azide-alkyne Huisgen cycloaddition.

Biomolecules (e.g., oligonucleotides) can be incorporated into the polymer coatings in a controlled manner, with particular biomolecules located at particular regions of the polymer coatings. Biomolecules can be incorporated into the polymer coatings at random, with particular biomolecules randomly distributed throughout the polymer coatings.

In some instances a composition of the invention comprises a surface, a polyacrylamide coating covalently bound to said surface; and at least one oligonucleotide coupled to said polyacrylamide coating. In other instances, the surface includes at least 1, 10, 100, 10,000, 100,000, 1,000,000, 10,000,000, 100,000,000, or 1,000,000,000 oligonucleotides coupled to the polyacrylamide coating.

Modification of Physicochemical Characteristics of Polymer Coating

The polymer coatings described in this disclosure can have their physicochemical characteristics modulated. This modulation can be achieved by incorporating modified acrylamide monomers during the polymerization process.

In some cases, ethoxylated acrylamide monomers can be incorporated during the polymerization process. Ethoxylated acrylamide monomers can be incorporated by being present in the polymerization solution. The ethoxylated acrylamide monomers can comprise monomers of the form CH₂═CH—CO—NH(—CH₂—CH2-O—)ₙH. The ethoxylated acrylamide monomers can comprise hydroxyethyl acrylamide monomers. The ethoxylated acrylamide monomers can comprise ethylene glycol acrylamide monomers. The ethoxylated acrylamide monomers can comprise hydroxyethylmethacrylate (HEMA). The ethoxylated acrylamide monomers can comprise N-(2-hydroxyethyl)acrylamide. The incorporation of ethoxylated acrylamide monomers can result in a more hydrophobic polyacrylamide surface coating.

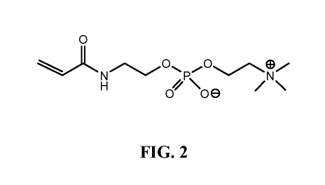

In some cases, phosphorylcholine acrylamide monomers can be incorporated during the polymerization process. The phosphorylcholine acrylamide monomers can comprise monomers of the structure shown in FIG. 2. The phosphorylcholine acrylamide monomers can comprise other phosphorylcholine acrylamide monomers. Phosphorylcholine acrylamide monomers can be incorporated by being present in the polymerization solution.

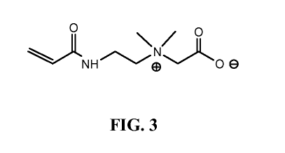

In some cases, betaine acrylamide monomers can be incorporated during the polymerization process. The betaine acrylamide monomers can comprise monomers of the structure shown in FIG. 3. Betaine acrylamide monomers can be incorporated by being present in the polymerization solution.

The polymer coating can be of uniform thickness. The polymer coating can be of varying thickness over its area. The polymer coating can be, on average, at least 1 μm thick. The polymer coating can be at least 2 μm thick. The polymer coating can be at least 3 μm thick. The polymer coating can be at least 5 μm thick. The polymer coating can be at least 10 μm thick. The polymer coating can be at least 15 μm thick. The polymer coating can be at least 20 μm thick. The polymer coating can be at least 25 μm thick. The polymer coating can be at least 30 μm thick. The polymer coating can be at least 40 μm thick. The polymer coating can be at least 50 μm thick. The polymer coating can be at least 75 μm thick. The polymer coating can be at least 100 μm thick. The polymer coating can be at least 150 μm thick. The polymer coating can be at least 200 μm thick. The polymer coating can be at least 300 μm thick. The polymer coating can be at least 400 μm thick. The polymer coating can be at least 500 μm thick. The polymer coating can be between about 1 μm and about 10 μm thick. The polymer coating can be between about 5 μm and about 15 μm thick. The polymer coating can be between about 10 μm and about 20 μm thick. The polymer coating can be between about 30 μm and about 50 μm thick. The polymer coating can be between about 10 μm and about 50 μm thick. The polymer coating can be between about 10 μm and about 100 μm thick. The polymer coating can be between about 50 μm and about 100 μm thick. The polymer coating can be between about 50 μm and about 200 μm thick. The polymer coating can be between about 100 μm and about 30 μm thick. The polymer coating can be between about 100 μm and about 500 μm thick.

Reactions

The polymer coatings described in this disclosure can be used in performing reactions. The reactions performed can be enzymatic. The reagents for the reactions performed can comprise nucleic acids. The reactions can comprise digestion reactions. The reactions can comprise extension reactions such as primer extension, or overlap extension. The reactions can comprise amplification reactions, such as polymerase chain reaction (PCR) and variants thereof (such as multiplex PCR, nested PCR, reverse transcriptase PCR (RT-PCR), semi-quantitative PCR, quantitative PCR (qPCR) or real time PCR, touchdown PCR, or assembly PCR), nucleic acid sequence based amplification (NASBA) (see e.g., “Compton, J (1991). Nucleic acid sequence-based amplification. Nature 350 (6313): 91-2.”), strand displacement assay (SDA) (see e.g., U.S. Pat. No. 5,712,124, “Strand displacement amplification”), and loop mediated isothermal amplification (LAMP) (see e.g., U.S. Pat. No. 6,410,278, “Process for synthesizing nucleic acid”). The reactions can comprise transcription reactions, such as in vitro transcription. The reactions can comprise sequencing reactions, such as BAC-based sequencing, pyrosequencing, sequencing by synthesis, or any method described in “Mardis, E. R. (2008). Next-generation DNA sequencing methods. Annu. Rev. Genomics Hum. Genet., 9, 387-402.”

The polymer coatings described in this disclosure can be robust. The robustness of the polymer coatings can be exhibited by the durability, the resistance to degradation, or the level of attachment of the coating after being subjected to certain conditions. The robustness of the polymer coatings can be exhibited by the number or percentage of biomolecules (e.g., oligonucleotides) molecules coupled to the polymer coating which remain coupled to the polymer coating after being subjected to certain conditions. Conditions can include but are not limited to duration of time, a temperature or set of temperatures, presence of chemicals (e.g., acids, bases, reducing agents, oxidizing agents), mechanical forces (e.g. stress, strain, vibrations, high pressures, vacuums), combinations of conditions, or repeated cycles of conditions or combinations of conditions (e.g. reaction cycles comprising temperatures and use of chemicals). Durations of time can comprise at least 1, 2, 3, 4, 5, 6, 7, 8, 9, 10, 20, 30, 40, or 50 minutes, at least 1, 2, 3, 4, 5, 6, 7, 8, 9, 10, 11, 12, 13, 14, 15,16, 17, 18, 19, 20, 21, 22, or 23 hours, at least 1, 2, 3, 4, 5, 6, 7, 8, 9, 10, 11, 12, or 13 days, or at least 2, 3, 4, 5, 6, 7, 8, 9, 10, 12, 14, 16, 18, 20, 22, 24, 26, 28, 30, 40, 50, or 60 weeks. Temperatures can comprise at least 0, 5, 10, 15, 20, 25, 30, 35, 40, 45, 50, 55, 60, 65, 70, 75, 80, 85, 90, 95, or 100° C. Temperatures can comprise at most 0, 5, 10, 15, 20, 25, 30, 35, 40, 45, 50, 55, 60, 65, 70, 75, 80, 85, 90, 95, or 100° C. Chemicals can comprise strong acids, weak acids, strong bases, weak bases, strong oxidizers, weak oxidizers, strong reducers, weak reducers, enzymes, monomers, polymers, buffers, solvents, or other reagents. Cycles of conditions can comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 125, 150, 175, 200, 250, 300, 350, 400, 450, 500, 600, 700, 800, 900, 1000, 2000, 3000, 4000, 5000, 6000, 7000, 8000, 9000, or 10,000 cycles. In some embodiments, the polymer coatings herein are used to perform at least 10, 20, 30, 40, 50, 60, 70, 80, 90, 100, 200, 300, 400, 500, 600, 700, 800, 900, or 1000 cycles of conditions, and wherein at least 50, 60, 70, 80, 90, 91, 92, 93, 94, 95, 96, 97, 98, 99, 99.5 or 99.9% the polymer chains remain completely intact and bonded to said surface after the cycles.

In some embodiments, the polymer coatings herein are used as a solid support to perform sequencing by synthesis (SBS). In SBS, a target polynucleotide sequence can be determined by generating its complement using the polymerase reaction to extend a suitable primer, and characterizing the successive incorporation of bases that generate the complement. The target sequence is, typically, immobilized on a solid support. Each of the different bases A, T, G or C is then brought, by sequential addition, into contact with the target, and any incorporation events detected via a suitable label attached to the base. In contrast to the prior art methods, the present invention requires the presence of a polymerase enzyme that retains a 3′ to 5′ exonuclease function, which is induced to remove an incorporated labeled base after detection of incorporation. A corresponding non-labeled base can then be incorporated into the complementary strand to allow further sequence determinations to be made. Repeating the procedure allows the sequence of the complement to be identified, and thereby the target sequence also. In some embodiments, the polymer coatings herein are used to perform at least 10, 20, 30, 40, 50, 60, 70, 80, 90, 100, 200, 300, 400, 500, 600, 700, 800, 900, or 1000 cycles of sequencing by synthesis (SBS), for example as described by the methods of U.S. Pat. No. 6,833,246, and wherein at least 50, 60, 70, 80, 90, 91, 92, 93, 94, 95, 96, 97, 98, 99, 99.5 or 99.9% the polymer chains remain completely intact and bonded to said surface after the SBS. Prior to the SBS cycles, the polymer coating can have coupled to it at least 10, 20, 50, 100, 200, 500, 1,000, 2,000, 5,000, 10,000, 20,000, 50,000 or 100,000, 200,000, 500,000, 1,000,000, 2,000,000, 5,000,000, 10,000,000, 20,000,000, 100,000,000, 200,000,000, 500,000,000, or a billion nucleic acid molecules. Prior to the SBS cycles, the polymer coating can have nucleic acid molecules arranged on it at an areal density of at least about 10, 20, 50, 100, 200, 500, 1,000, 2,000, 5,000, 10,000, 20,000, 50,000, 100,000, 1,000,000, 1×10⁷, 5×10⁷, 1×10⁸, 5×10⁸, 1×10⁹, 5×10⁹, 1×10¹⁰, 5×10¹⁰, or 1×10¹¹ molecules per square micrometer. In some cases, prior to the SBS cycles, the polymer coating has nucleic acid molecules arranged on it at an areal density of about 1×10² to about 1×10⁶ per square micrometer. In some cases, prior to the SBS cycles, the polymer coating has nucleic acid molecules arranged on it at an areal density of about 5×10² to about 5×10⁴ per square micrometer. In some cases, prior to the SBS cycles, the polymer coating has nucleic acid molecules arranged on it at an areal density of about 1×10³ to about 1×10⁴ per square micrometer.

In some embodiments, the polymer coatings herein are used to perform PCR on nucleic acid polymer chains bound to the coating. PCR, for example, can include multiple cycles, wherein each cycle includes a denaturation step, an annealing step, and an extension or elongation step. The denaturation step can comprise subjecting the nucleic acids to a temperature of at least about 85° C., 86° C., 87° C., 88° C., 89° C., 90° C., 91° C., 92° C., 93° C., 94° C., 95° C., 96° C., 97° C., or 98° C. The denaturation step can comprise duration of at least about 15 seconds, 20 seconds, 25 seconds, 30 seconds, 35 seconds, 40 seconds, or 45 seconds. The annealing step can comprise subjecting the nucleic acids to a temperature of at least about 50° C., 51° C., 52° C., 53° C., 54° C., 55° C., 56° C., 57° C., 58° C., 59° C., 60° C., 61° C., 62° C., 63° C., 64° C., or 65° C. The annealing step can comprise duration of at least about 15 seconds, 20 seconds, 25 seconds, 30 seconds, 35 seconds, 40 seconds, or 45 seconds. The extension or elongation step can comprise a temperature of at least about 70° C., 71° C., 72° C., 73° C., 74° C., 75° C., 76° C., 77° C., 78° C., 79° C., or 80° C. The extension or elongation step can comprise duration of at least about 30 seconds, 40 seconds, 50 seconds, 60 seconds, 70 seconds, 80 seconds, 90 seconds, 100 seconds, 110 seconds, or 120 seconds. The polymer coatings herein can be used to perform at least 10, 20, 30, 40, 50, 60, 70, 80, 90, or, 100 cycles of polymerase chain reaction (PCR), and wherein at least 50, 60, 70, 80, 90, 91, 92, 93, 94, 95, 96, 97, 98, 99, 99.5 or 99.9% the polymer chains remain completely intact and bonded to said surface after the final PCR cycle. Prior to the PCR cycles, the polymer coating can have coupled to it at least 10, 20, 50, 100, 200, 500, 1,000, 2,000, 5,000, 10,000, 20,000, 50,000 or 100,000, 200,000, 500,000, 1,000,000, 2,000,000, 5,000,000, 10,000,000, 20,000,000, 100,000,000, 200,000,000, 500,000,000, or a billion nucleic acid molecules. Prior to the PCR cycles, the polymer coating can have nucleic acid molecules arranged on it at a density of at least 10, 20, 50, 100, 200, 500, 1,000, 2,000, 5,000, 10,000, 20,000, 50,000, 100,000, 1,000,000, 1×10⁷, 5×10⁷, 1×10⁸, 5×10⁸, 1×10⁹, 5×10⁹, 1×10¹⁰, 5×10¹⁰, or 1×10¹¹ molecules per square micrometer. In some cases, prior to the PCR cycles, the polymer coating has nucleic acid molecules arranged on it at an areal density of about 1×10² to about 1×10⁶ per square micrometer. In some cases, prior to the PCR cycles, the polymer coating has nucleic acid molecules arranged on it at an areal density of about 5×10² to about 5×10⁴ per square micrometer. In some cases, prior to the PCR cycles, the polymer coating has nucleic acid molecules arranged on it at an areal density of about 1×10³ to about 1×10⁴ per square micrometer.

Advantages

Use of initiator species, such as silanes, with multiple bonding groups can provide high thermal and hydrolytic stability (see, e.g., U.S. Pat. No. 6,262,216). Such stability can increase the durability of the coating through repeated cycles of reactions or other processing.

Use of surface coatings as described herein can provide a more enzymatically compatible or favorable environment than that provided by an uncoated surface. Surface coatings with modulated physicochemical characteristics as described herein can provide advantages to use for conducting enzymatic reactions on, near, or on molecules bound to the surfaces. The advantages can comprise a reduction in non-specific binding to the surface. The advantages can comprise an optimal environment for enzymes, such as polymerases. For example, neutral hydrophilic polymers and linking groups can provide favorable environments for enzymes.

### EXAMPLES

### Example 1

### Production of a Flat Surface Array

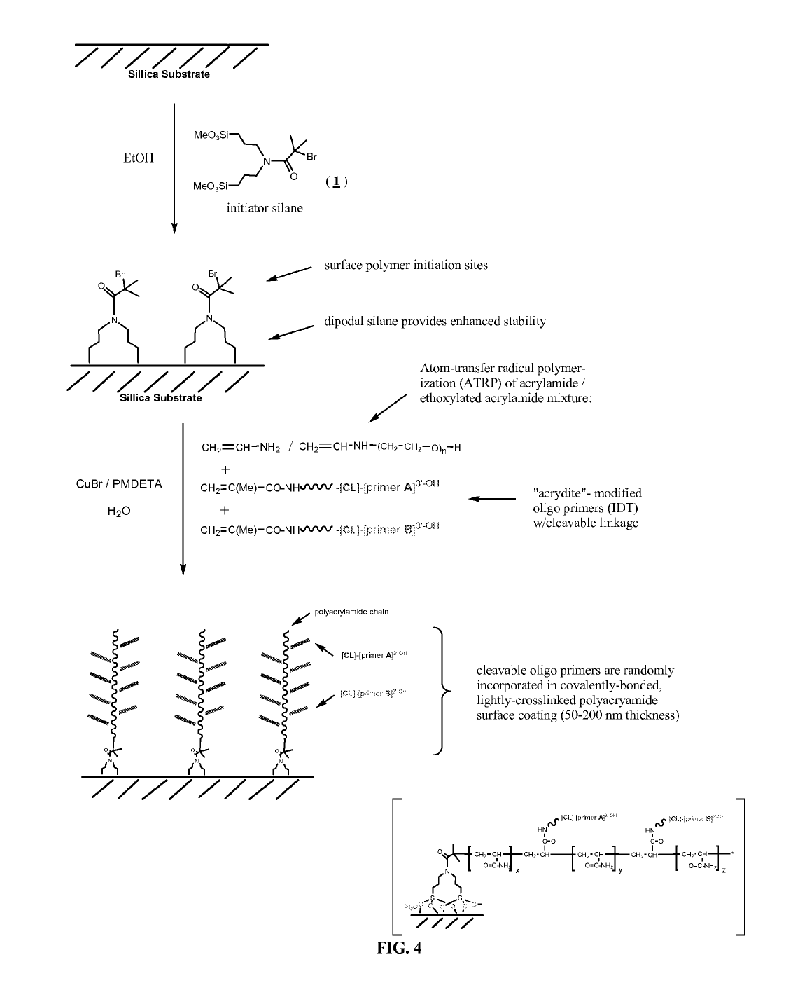

Initiator silanes of the structure shown in FIG. 1 are bound to a flat silica substrate in the presence of EtOH, forming di-podal surface polymer initiation sites. A mixture of acrylamide and ethoxylated acrylamide, together with acrydite-modified oligonucleotides, undergoes atom-transfer radical polymerization (ATRP) on the substrate in the presence of CuBr, PMDETA, and H₂O. This forms a covalently-bonded, lightly-crosslinked polyacrylamide surface coating bound to the surface initiator sites, with thickness between about 50 nm and about 200 nm, with oligonucleotides incorporated into the structure (see FIG. 4).

### Example 2

### Use of a Flat Surface Array in Sequencing

A polyacrylamide coated substrate is prepared as described in Example 1. DNA to be sequenced is bound to the oligonucleotides incorporated into the polymer structure. Sequencing by synthesis reagents are added to the substrate and sequencing by synthesis is performed for 40 cycles. At least 90% of polymer chains remain intact and bonded to the surface.

### Example 3

### Use of a Flat Surface Array in DNA Amplification

A polyacrylamide coated substrate is prepared as described in Example 1. DNA to be amplified is bound to the oligonucleotides incorporated into the polymer structure. Polymerase chain reaction (PCR) reagents are added to the substrate and PCR is performed for 30 cycles. At least 90% of polymer chains remain intact and bonded to the surface.

### Example 4

### Synthesis of Azido-PEG4-N,N-bis(3-(trimethoxysilyl)propyl)carbamate

Azido-PEG4-alcohol (BroadPharm, 220 mg; 1.0 mmol) was dried by co-evaporating twice with 2 ml CH₃CN, then combined with diphosgene (200 mg; 1.0 mmol) in 1 ml of CH₂Cl₂ under N2. After standing overnight at ambient temperature, the solvent was evaporated to obtain 280 mg of the product as a pale yellow oil, which was used without further purification. ¹H-NMR (CDCl₃): δ(ppm) 4.46 (2H, t J=2.8 Hz; CH₂OC(O)CI); 3.79 (2H, t J=4.5 Hz; CH₂CH₂N₃); 3.68-3.70 (10H, m, CH₂OCH₂); 3.41 (2H, t J=5.2 Hz, CH₂N₃).

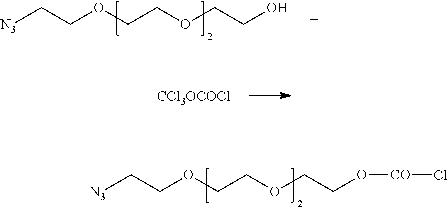

Bis(trimethoxysilylpropyl)amine (342mg/320 uL; 1.0 mmol) and DIEA (136 mg/182 uL; 1.05 mmol) were combined in 1 ml dry ether under N₂ and cooled on ice to 0-4° C. The azido-PEG4 chloroformate (280 mg; 1.0 mmol) was dissolved in lml dry ether and added dropwise via syringe, and then stirring was continued at ambient temperature overnight. Another 2 ml of dry ether was added, and the solution was quickly filtered and evaporated to yield the silane as a light yellow oil (˜550 mg). ¹H-NMR (CD₃OD): δ(ppm) 4.20-4.24 (2H, br m, CH₂OC(O)N<); 3.67-3.74 (13H, m, CH₂OCH₂); 3.39 (2H, t J˜5.0 Hz, CH₂N₃); 3.35 (21H, s, CH₃OSi); 3.22-3.28 (4H, br m, —CH₂NC(O)O—); 1.60-1.70 (4H, br m, C—CH₂—C); 0.55-0.65 (4H, br m, C—CH₂—Si).

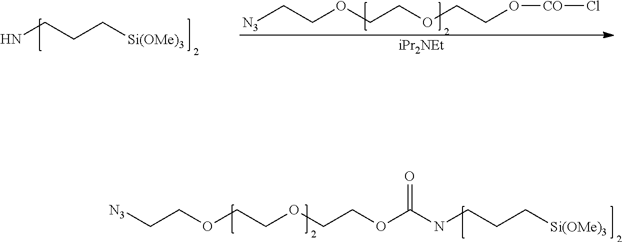

### Example 5

### Synthesis of N-(3-(Bromoacetamido)propyl)methacrylamide

N-(3-aminopropyl)methacrylamide hydrochloride (Polysciences; 360 mg; 2.0 mmol) and N-(bromoacetoxy)succinimide (Broad Pharm; 570 mg; 2.4 mmol) were combined in 10 mL dry CH₂Cl₂ under N₂ and cooled to −10° C. with ice-MeOH. Diisopropylethylamine (Aldrich, 800 uL; 4.2 mmol) was then added dropwise while stirring. The solution was stirred for another 30 min cold, then for 3 h at rm temp. The solution was diluted with 40 ml ethyl acetate, and washed successively with 12 ml each of 1M HC1; 0.1M NaOH; and then brine. The organic phase was dried with MgSO₄ and evaporated to yield 220 mg (˜40%) of 3:1 mixture of bromo-, and chloroacetylated products as an off-white solid. ¹H-NMR (acetone-d₆): δ(ppm) 7.70 (1H, br s, NHₐ); 7.40 (1H, br s, NH_{b}); 5.71-5.73 (1H, br m, CH═C); 5.30-5.32 (1H, m, CH′═C); 4.08 (0.5H, s, CH₂C1); 3.89 (1.5H, s, CH₂Br); 3.24-3.32 (4H, m, CH₂N); 1.91-1.93 (3H, br m, CH₃); 1.68 (2H, br qnt, J=6.4 Hz; H₂′CCH₂CH₂″). LC-MS (ESI): 5.7 min: 242, 243, 244 (10:1:3; M.Na⁺/chloro); 219, 220, 221 (10:1:3; M.H⁺chloro); 134, 135, 136 (10:0.6:3; M-CH₂═C(Me)CONH⁻/chloro); 126, 127 (10:1; M-Cl/BrCH₂CCONH⁻). 5.9 min: 286, 287, 288, 289 (10:1:10:1; M.Na⁺/bromo); 263, 265, 266 (10:10:1; M.H⁺/bromo); 178, 179, 180, 181 (10:0.6:10:0.6; M-CH₂═C(Me)CONH⁻/bromo); 126, 127 (10:1; M-Cl/BrCH₂CCONH⁻).

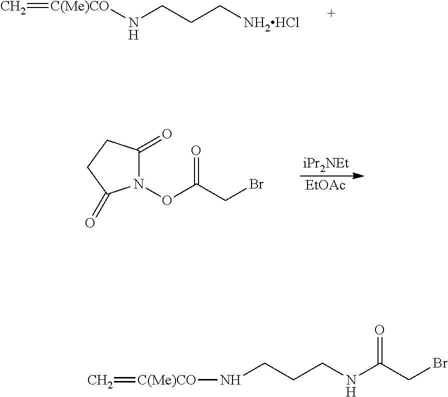

### Example 6

### Synthesis of N-(4-Azidobutyl)methacrylamide

4-Azido-1-butylamine (Synthonix; 1.1 g; 8.75 mmol)) was combined with DIEA (1.22 g; 9.5 mmol) in 15 mL of dry ethyl acetate in a 50 mL flask equipped w/stirbar & dropping funnel and flushed with dry N₂. The solution was cooled to 2° C. on an ice-waterbath, and a solution of methacryoyl chloride (0.96 g; 9.2 mmol) in 5 ml dry ether was added dropwise with stirring over 30 min. The ice bath was removed, another 15 ml of dry ethyl acetate was added, and stirring was continued at ambient temperature overnight. The solids were removed by filtration and the combined filtrates were washed twice w/10 ml water, once w/brine, then dried (MgSO₄) evaporated in vacuo to obtain1.50 g (93%) product as an orange liquid. ¹H-NMR (CDCl₃): δ(ppm) 5.92-5.83 (1H, br s, NH); 5.68 (1H, t J=0.8 Hz; ═CHₐ); 5.68 (1H, m, ═CH_{b}); 3.45 (4H, br m, NCH₂); 1.97 (3H, t J=1.4 Hz, CH₃); 1.69-1.60 (4H, br m, C—CH₂—C). MS (ESI): 126.2 (M-CH₂N₃); 183.2 (M.H+); 205.2 (M.Na+). The product was used within 10 days, as decomposition with evolution of N₂ was noted after 2-3 weeks storage at 4° C. by NMR.

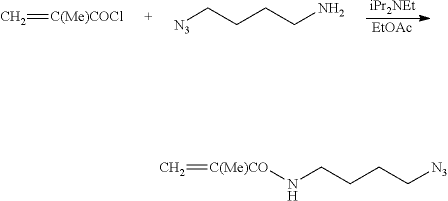

### Example 7

### Silanation of Flowcell Surfaces

For most experiments, the flowcells used were flat “capillary micro glass slides” made from Corning® 7740 borosilicate, low expansion, type I glass (p/n 63825-05, EM Sciences, Hatfield, Pa.). A short length of 0.5 mm ID heat-shrink PTFE tubing was sealed to both ends of the capillaries to provide leak-proof connection to manifolds, syringes, etc. For some experiments, “refurbished” Illumina MiSeg™ flowcells were employed. These were stripped of indigenous surface coatings with 200 mM sodium persulfate at 65° C. for 18 hr, followed by 1M KOH/65° C./6 hr, rinsing with deionized water and drying with a stream of nitrogen.

Prior to silanation, all capillary flowcell surfaces were cleaned by immersion in sulfuric-peroxide solution (Nanostrip, Cyantek Corp., Fremont Calif.) for 16-18 hr at 25° C., then rinsed thoroughly with deionized water and dried with a stream of nitrogen. The cleaned flowcells were stored under nitrogen and silanated within 48 hours. Silanation was performed by filling the flowcell with a freshly prepared 2% (wt/vol) solution of the appropriate silane in 95:5 ethanol-water, and incubating for 4-18 hours at room temperature. The flowcells were then rinsed thoroughly with ethanol and deionized water; dried with nitrogen, and stored at ambient temperature.

### Example 8

### Oligonucleotide Primer Immobilization by Surface-Initiated Acrylamide ATRP

Flowcells for SI-ATRP were silanated as described in Example 7, with 2-Bromo-2-methyl-N,N-bis-(3-trimethoxysilanylpropyl)propionamide (see, e.g., US 2011/0143967).

Dry-down Primers: Equivalent amounts of 5′-acrydite modified primers FWD (4 uL, 1 mM) and REV (4 uL, 1 mM) were combined in a 0.9 mL conical-tip HPLC vial. The solutions were reduced to dryness on a Speed-Vac evaporator at ambient temperature (10-15 minutes). The vial containing dried primers was tightly closed with a septum-sealed screw cap and connected to a vacuum/N2 manifold via an 18-guage syringe needle. The vial was deoxygenated 5 cycles of alternating vacuum/nitrogen refill through a syringe needle.

Deoxygenate Flowcell: The flowcell to be used for Sl-ATRP was deoxygenated by purging with dry nitrogen.

Deoxygenate Solvent: In another vial, a solvent mixture composed of 28% methanol in water (v:v) was deoxygenated by sparging continuously with nitrogen for 30 minutes,

Preparation of Catalyst/Acrylamide Solution: CuBr (6.8 mg, 47.4 umol) and CuBr₂ (3.9 mg, 17.5 umol) were weighed and placed in a 20 mL septum-capped vial containing a magnetic stirring bar. The vial was connected to a vacuum/nitrogen manifold and deoxygenatedcarefully with three cycles of evacuation-nitrogen back-fill. Then a portion of the deoxygenated solution (14.5 mL) was transferred to the vial containing the copper salts via gas-tight syringe. Finally, acrylamide (42.5 mg, 600 umol) and PMDETA (14 uL, 67.2 umol) were added, and the solution was stirred vigorously while sparging with nitrogen for another 15 minutes. It was occasionally necessary to sonicate the solution briefly to disperse the CuBr solid to obtain a light blue homogeneous solution.

Transfer Polymerization Solution to Flowcell: The dried-down primers were reconstituted in deoxygenated catalyst/acrylamide solution (20 uL), which was transferred via gastight syringe. The resulting solution was transferred to the pre-purged flowcell from step 3, filling it completely. The ends of the flowcell were sealed with parafilm, and the flowcell was maintained at ambient temperature for 24-48 hours in an anaerobic environment.

Wash and Storage: The flowcell was flushed with 28% methanol-water), and 1×E buffer (˜1 mL/ea) and stored at 4° C.

### Example 9

### Oligonucleotide Primer Immobilization Via Solution-Initiated FRP Grafting of acrylamide/bromoacetyl-acrylamide

Flowcell surfaces were silanated with 3-(acrylamido)propyltrimethoxysilane (Gelest, Inc).

Purge Flowcell: The flowcell to be used for FRP was deoxygenated by purging with dry nitrogen.

Solution Preparation and Polymerization: A solution of acrylamide (0.0713 g, 1 mmol) and N-(3-bromoacetamidopropyl)methacrylamide (6.4 mg, 0.024 mmol) in Milliq water (5 g) in a vial was capped with rubber septum-sealed cap. The solution was deoxygenated by sparging with nitrogen for 30 minutes. Polymerization was initiated by adding a solution of potassium persulfate (2.5 mg, 0.0093 mmol in degassed water 50 uL) and neat tetramethylenediamine (4.45 mg, 0.038 mmol). The resulting solution was transferred immediately into the flowcell, filling it completely. The ends of the flowcell were sealed with parafilm, and the flowcell was maintained at ambient temperature for 60-80 minutes in an anaerobic environment. Polymerization was terminated by purging the flowcell with 4-6 mL of water, followed by 1 mL of 6×SSPE to remove unbound polymer. The flowcell was stored in 6×SSPE at 4° C.

Primer Conjugation: A combined solution of FWD (2.5 uL, 1 mM) and REV (2.5 uL, 1 mM) 5′-phosphorothioate-modified primers was placed in a 0.9 mL conical-tip HPLC vial. The solution was reduced to dryness on a Speed-Vac evaporator at ambient temperature (10-15 minutes) and then redissolved in 6×SSPE (20 uL). The storage solution was removed from the flowcell and replaced with the primer solution via a gas-tight syringe. The ends of the flowcell were sealed tightly with parafilm, and the flowcell was maintained at 55° C. for 2 hours. The flowcell was allowed to cool to ambient temperature and then rinsed with Milliq water, 6×SSPE, and 1×TE (1 mL per rinse). The flowcell containing 1×TE was sealed with parafilm and stored at 4° C.

### Example 10

### Direct Immobilization of Primers on Silanated Flowcell Surface Using Click Chemistry

Flowcell surfaces were cleaned by immersion in sulfuric-peroxide solution (Nanostrip, Cyantek Corp., Fremont Calif.) for 16-18 hr at 25° C., then rinsed thoroughly with deionized water and dried with a stream of nitrogen. Flowcells were stored under nitrogen and silanated within 48 hours with a freshly prepared 2% (wt/vol) solution of Azido-PEG4-N,N-bis(3-(trimethoxysilyl)propyl)carbamate in 95:5 ethanol-water for 18 hours. The flowcells were then rinsed thoroughly with ethanol and deionized water; and dried with nitrogen. A solution containing 100 uM each of the 5′-alkynyl-modified oligonucleotide primers FWD and REV, 5 mM CuI, and 10 mM tris-(3-hydroxypropyltriazolylmethyl)amine (THPTA) in 0.1M Tris buffer (pH 7.0) was added and maintained at 22° C. for 18 hours, after which the oligonucleotide solution was removed and the flowcell was rinsed with deionized water, dried & stored at 4° C.

### Example 11

### Immobilization Analysis by Hybridization

Successful primer attachment was confirmed with a 5′-CY3-labeled oligonucleotide hybridization target complimentary to the FWD primer (“FWD”): the flowcell was filled with 250 nM target oligo in 6×SSPE buffer pH 7.4, incubated for 1 h at 55° C., cooling to 25° C., and then washed with 4-5 volumes 6×SSPE. Surface fluorescence was measured with a CCD-based imaging fluorescence microsope (LED bb excitation; >640 nm emission filter). The hybridization target solution was then removed and the flowcell was washed out with 20 volumes of formamide at 55° C., and stored at 4° C. in nuclease-free water.

### Example 12

### Solid Phase DNA Amplification and Cluster Generation

Prepared flowcells (e.g., those prepared in previous examples) were placed on a programmable thermo-fluidic station (purpose built CentiPD). An actively cooled Peltier thermoelectric module (Laird), NTC thermistor temperature sensors and a programmable PID Controller (Laird) provided thermal control. The range of achievable temperatures was 20-100° C. On the fluidic side, a 250 ul syringe pump (Cavro) pulled a programmed volume of reagent at a specified speed through the capillary flowcell. The appropriate reagent was selected via a 24-way selector valve (VICI) with sippers leading to each of the reagent tubes. The prepared reagents Eppendorf tubes were sitting in an aluminum cooling block placed in an ice bath (to maintain them at 4° C. during the protocol time period).

A solution of 10 mM dNTPs was prepared as follows: combine 300 μL of each dNTP stock solution (stock solution concentration: 100 mM) to make 25 mM stock, then add 1000 μL of 25 mM stock to 1500 μL of 10 mM Tris pH 8.0.

An HB1 solution was prepared in 1× (˜10 mL aliquot) and 5× amounts, shown in Table 1:

TABLE 1
HB1 solution

| Reagent | Stock | Final | 1 RXN | |
|---|---|---|---|---|
| H2O | | | 7400 | ul |
| 20X SSC | 20X | 5X | 2500 | ul |
| Tween-20 | 10% | 0.1% | 100 | ul |
| Total | | | 10 | ml |

A Wash Buffer (W2) solution was prepared in 5× and 1× amounts (˜10 mL aliquot), as shown in Table 2:

TABLE 2
W2 solution

| Reagent | Stock | Final | 1 RXN | | 5 RXNS | |
|---|---|---|---|---|---|---|
| H2O | | | 9750 | ul | 48750 | ul |
| 20X SSC | 20X | 0.3X | 150 | ul | 750 | ul |
| Tween-20 | 10% | 0.1% | 100 | ul | 500 | ul |
| Total | | | 10 | ml | 50000 | ul |

Labeled Primer (FP) solution was prepared at a concentration of 5 μM by adding 15 μL of 500 μM primer stock solution to 1485 HB1 solution as shown in Table 3:

TABLE 3
FP solution

| Reagent | Stock | Final | 1 RXN | | 16 RXNS | | Cost |
|---|---|---|---|---|---|---|---|
| HB1 | | | 360 | ul | 5760 | ul | $0 |
| 5 uM Primer | 5.0 uM | 0.5 uM | 40 | ul | 640 | ul | $0 |
| Total | | | 400 | ul | 6400 | ul | $0 |

An Amplification Premix (APM) solution was prepared as shown in Table 4:

TABLE 4
APM Buffer solution

| | | | | |
|---|---|---|---|---|
| Reagent | Stock | Final | 1 RXN | 32 RXNS |

| | | | | | | |
|---|---|---|---|---|---|---|
| H2O | | | 687 | ul | 21984 | ul |
| 10X Thermopol | 10X | 1X | 100 | ul | 3200 | ul |
| 5M Betaine | 5M | 1M | 200 | ul | 6400 | ul |
| DMSO | 100% | 1.3% | 13 | ul | 416 | ul |
| Total | | | 1000 | ul | 32000 | ul |

An Amplification Mix (AM) was prepared as shown in Table 5:

TABLE 5
AM Buffer solution

| Reagent | Stock | Final | 1 RXN | | 16 RXNS | | Cost |
|---|---|---|---|---|---|---|---|
| H2O | | | 1756 | ul | 28090 | ul | $0 |
| 10X | 10X | 1X | 280 | ul | 4480 | ul | $0 |
| Thermopol | | | | | | | |
| 5M Betaine | 5M | 1M | 560 | ul | 8960 | ul | $128 |
| DMSO | 100% | 1.3% | 36 | ul | 582 | ul | $0 |
| 10 mM dNTPs | 10 mM | 0.2 mM | 56 | ul | 896 | ul | $0 |
| Bst Lg. | 8 U/ul | 0.32 U/ul | 112 | ul | 1792 | ul | $444 |
| Fragment | | | | | | | |
| Total | | | 2800 | ul | 44800 | ul | $573 |

An Linearization Mix (LM) solution was prepared as shown in Table 6:

TABLE 6
LM solution

| Reagent | Stock | Final | 1 RXN | | 16 RXNS | | Cost |
|---|---|---|---|---|---|---|---|
| H2O | | | 356 | ul | 5696 | ul | $0 |
| 10X Thermopol | 10X | 1X | 40 | ul | 640 | ul | $0 |
| USER | 1 U/ul | 0.01 U/ul | 4 | ul | 64 | ul | $83 |
| Total | | | 400 | ul | 6400 | ul | $83 |

A Library Dilution Buffer was prepared which comprises 10 mM Tris-Cl at pH 8.5 with 0.1% Tween-20.

A dilute library was prepared as follows: 1) Stock 2 N NaOH solution was diluted to 0.1 N NaOH solution, as shown in Table 7. 2) Stock 10 nM PhiX solution was diluted to 2 nM by adding 2 μL of PhiX to 8 μL of Library Dilution Buffer. 3) The sample was denatured by adding 10 μL of 0.1 N NaOH to 10 μL of 2 nM sample solution and incubating for 5 minutes at room temperature. 4) The denatured sample was diluted to 20 pM by adding 980 μL of pre-chilled HB1 solution to 20 μL of sample. 5) The diluted sample was further diluted to 7 pM by adding 650 μL of pre-chilled HB1 solution to 350 μL of 20 pM sample solution. 6) The diluted sample was saved on ice until later use.

TABLE 7
0.1N NaOH solution

| | | |
|---|---|---|
| Reagent | 1 RXN | 4 RXNS |

| | | | | |
|---|---|---|---|---|
| H2O | 475 | ul | 1900 | ul |
| 2N NaOH | 25 | ul | 100 | ul |
| Total | 500 | ul | 2000 | ul |

A reagent plate was loaded with solutions in 2 mL Eppendorf tubes, with reagent tubes matched to appropriate CentPD sippers, as follows: Reagent 1: 950 ul HB1; Reagent 2: 950 ul APM; Reagent 3: 1300 ul AM1; Reagent 4: 1100 ul FM (Formamide 100%); Reagent 5: 1300 ul AM2; Reagent 6: 1100 ul W2; Reagent 7: 350 ul LM; Reagent 8: 400 ul NAOH (0.1 N NaOH); Reagent 9: 400 ul FP.

A prepared flowcell, such as described in previous examples, was placed on a thermo-fluidic station and a clustering protocol was initiated and run on a CentPD as described in Table 9:

TABLE 9
CentPD clustering protocol

| | | Heat Step | | | Wait | Flow Step | | | |
|---|---|---|---|---|---|---|---|---|---|
| | | Temp | Rate | Time | Time | | Volume | Rate | Time |
| Flow Check | Repeats | [° C.] | [° C./s] | [s] | [s] | Chem | [μL] | [μL/s] | [s] |
| Initial Prime | | 25 | | | 60 | HB1 | 60 | 4 | 15 |
| | | | | | | W2 | 60 | 4 | 15 |
| | | | | | | NAOH | 60 | 4 | 15 |
| | | | | | | APM | 60 | 4 | 15 |
| | | | | | | AM1 | 60 | 4 | 15 |
| | | | | | | AM2 | 60 | 4 | 15 |
| | | | | | | FM | 60 | 1 | 60 |
| | | | | | | HB1 | 120 | 1 | 120 |
| TMP | | 90 | | | | Library | 150 | 1 | 150 |
| Introduction | | | | | | W2 | 20 | 1 | 20 |
| TMP | | 40 | 0.05 | 1120 | 80 | | | | |
| Rampdown | | | | | | | | | |
| TMP Buffer | | | | | | W2 | 100 | 0.5 | 200 |
| wash | | | | | | | | | |
| First | | | | | | W2 | 100 | 0.5 | 200 |
| Extension | | | | | | AIR | 3 | 0.5 | 6 |
| | | | | | | AM1 | 100 | 1 | 100 |
| | | | | | | AIR | 3 | 0.5 | 6 |
| | | | | | | W2 | 40 | 1 | 40 |
| FE Wait | | 40 | | | 90 | | | | |
| Amp- | | | | | | | | | |
| TempRamp | | | | | | | | | |
| Template | | 25 | | | | NAOH | 150 | 0.5 | 300 |
| Strip | | 60 | | | | W2 | 150 | 1 | 150 |
| Amplification 1 | 16X | | | | | FM | 28 | 8.5 | 8 |
| | | | | | | APM | 28 | 1 | 28 |
| | | | | | | AM1 | 72 | 4 | 18 |
| Amplification 2 | 16X | | | | | FM | 28 | 3.5 | 8 |
| | | | | | | APM | 28 | 1 | 28 |
| | | | | | | AM2 | 72 | 4 | 18 |
| Amplification | | 25 | | | | W2 | 120 | 2 | 60 |
| wash | | | | | | HB1 | 95 | 4 | 23.75 |
| Linearization | | 38 | | | 300 | LM | 150 | 1 | 150 |
| Start | | | | | | | | | |
| Linearization | 5X | | | | 300 | LM | 20 | 1 | |
| Cycle | | | | | | | | | |
| Linearization | | 25 | | | | W2 | 150 | 4 | 37.5 |
| Finish | | | | | | | | | |
| Read 1 | | 60 | | | 300 | HB1 | 95 | 4 | 23.75 |
| Preparation | | 40 | | | | NAOH | 200 | 1 | 200 |
| | | 25 | | | | W2 | 200 | 1 | 200 |
| | | | | | | FP | 200 | 1 | 200 |
| | | | | | | W2 | 150 | 1 | 150 |
| | | | | | | HB1 | 150 | 1 | 150 |

1) All the reagents were primed (60 μL, 4 μL/s 25 ° C.), last of which were HB1 and W2 buffers (Illumina nomenclature). 2) 150 μL of template was introduced at 90° C. at a rate of 1 μL/s. The template was a PhiX DNA library (7 pM in HB1, denatured, insert size 450 bp). 3) After incubating for 30 seconds, the temperature was slowly reduced to 40° C. over 18 minutes at a rate of 0.05 deg/s. 4) The excess template was washed out with 200 μL of W2 at 0.5 μL/sec, also at 40° C. 5) First extension of the grafted primers was achieved by infusing 150 μL of amplification mix (AM1) at 1 μL/s, book ended with 3 μL air bubbles in order to prevent mixing of reagents that may occur in the line in transit to the flowcell. A 90 second incubation step allows plentiful time for full template replication by Bst enzyme. 6) The flowcell is cooled to 25° C., and the template is stripped with 150 μL of 0.1N NaOH pumped at rate of 0.5 μL/s, followed by 150 μL of buffer (W2). 7) The flowcell is heated to 60° C. in preparation for isothermal amplification. 8) 32 cycles of isothermal amplification are performed by repeating these 3 steps: (a) denaturation in 100% formamide (FM) 28 μL/s at 3.5 μL/s; (b) pre-amplification buffer without the enzyme (APM) to remove formamide & allow for re-hybridization, 28 μL at 1 μL/s; and (c) extension of the primer with amplification mix (AM), 72 μL at 4 μL/s. 9) The amplification reagents are washed out with 120 μL of W2 and 95 μL of HB1). 10) 150 μL linearization reagent (LM) is introduced at 1 μL/s, temp 25° C. (to cut half of the amplified strands). 11) The flowcell is heated to 38° C., and incubated for 5 min (USER treatment, cutting of dU via Uracil DNA Glycosylase). 12) Fresh 20 μL of the LM solution is moved into the flowcell and incubating for 5 min, repeated five times. 13) After linearization, the temperature is reduced to 25° C., and washed with 150 μL W2 and 95 μL HB1. 14) The flowcell is denatured again with 200 μL of 0.1N NaOH and washed with 200 μL of W2. 15) Cy3 5′ labeled sequencing primer (FP) complimentary to the remaining strand is introduced 200 μL at 1 μL/s. 16) The temperature is raised to 60° C. and the solution is incubated for 5 min to allow for hybridization. 17) After reducing the temperature to 40° C., excess primer is washed away with 150 μL W2. 18) After further reducing the temperature to 25° C., the flowcell is further washed with 150 μL of W2.

Images of clustered colonies were taken on a custom epi-fluorescence microscope with an Alta U-4000 CCD camera (Apogee). Since the hybridized primers were labeled on the 5′ end with Cy3 fluorophore, we used Cy3-4040C filter cube (Semrock) and a 532 nm LED as the excitation light source. The images were magnified 40× with an ELWD Nikon 0.6 NA objective, rendering a field of view 375×375 um in size.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A composition, comprising:
a surface with a 10 or more nucleic acid molecules coupled thereto, wherein at least 90% of said nucleic acid molecules remain intact and coupled to said surface after at least 30 PCR cycles, wherein each PCR cycle comprises the following reaction conditions:
(a) a denaturation step at a temperature of at least 85° C. for at least 15 seconds;
(b) an annealing step at a temperature of at least 50° C. for at least 15 seconds; and
(c) an extension step at a temperature of at least 70° C. for at least 30 seconds.

2. The composition of claim 1, wherein the surface is covered with a polymer brush.

3. The composition of claim 2, wherein the polymer brush comprises acrylamide.

4. The composition of claim 3, wherein the polymer brush further comprises N-(2-hydroxyethyl)acrylamide.

5. The composition of claim 1 wherein at least 1,000 different nucleic acid molecules are coupled to said surface.

6. The composition of claim 1 wherein at least 100,000 different nucleic acid molecules are coupled to said surface.

7. The composition of claim 1 wherein at least 1,000,000 different nucleic acid molecules are coupled to said surface.

8. A method for performing an enzymatic reaction, comprising:
(a) providing a substrate having a polymer brush coating and a plurality of biomolecules coupled to said polymer brush; and
(b) performing one or more enzymatic reactions with said biomolecules on said substrate.

9. The method of claim 8, wherein the biomolecules are selected from the group consisting of: oligonucleotides, polynucleotides, aptamers, proteins, and antibodies.

10. The method of claim 9, wherein the enzymatic reaction is selected from the group consisting of: polymerase chain reaction, sequencing reaction, ligation reaction, extension reaction, and transcription reaction.

11. The method of claim 9, further comprising applying heat to said substrate.

12. The method of claim 9, wherein at least 90% of said biomolecules are retained with at least 90% integrity after 40 cycles of sequencing by synthesis reactions.

13. The method of claim 9, wherein at least 90% of said biomolecules are retained with at least 90% integrity after 25 cycles of polymerase chain reactions.

14. The method of claim 9, wherein the substrate comprises at least 1,000,000 different types of biomolecules, and wherein each biomolecule is an oligonucleotide.

15. The method of claim 14, wherein said enzymatic reaction is an extension reaction.

16. A method for making a modified surface, comprising:
(a) providing a surface;
(b) covalently bonding initiator species to said surface;
(c) conducting surface initiated polymerization of a polymer from said initiator species, thereby producing a polymer coating comprising a plurality of polymer chains; and
(d) coupling two or more different biomolecules to said polymer coating.

17. A method for making a modified surface, comprising:
(a) providing a surface;
(b) covalently bonding initiator species to said surface;
(c) conducting surface initiated polymerization of a mixture two or more different types of acrylamide monomers from said initiator species, thereby producing a polymer coating comprising a plurality of polymer chains; and
(d) coupling biomolecules to said polymer coating.

18. The method of claim 16 or 17, wherein the biomolecules are selected from the group consisting of: oligonucleotides, polynucleotides, aptamers, proteins, and antibodies.

19. The method of claim 16, wherein the two or more different biomolecules are two different oligonucleotides.

20. The method of claim 17, wherein the two or more different types of acrylamide monomers are selected from the group consisting of: acrylamide, N-(2-hydroxyethyl)acrylamide, ethylene glycol acrylamide, and hydroxyethylmethacrylate (HEMA).

21. The method of claim 16 or 17, wherein said surface is selected from the group consisting of glass, silica, titanium oxide, aluminum oxide, indium tin oxide (ITO), silicon, polydimethylsiloxane (PDMS), polystyrene, polycyclicolefins, polymethylmethacrylate (PMMA), titanium, and gold.

22. The method of claim 16 or 17, wherein said surface comprises glass.

23. The method of claim 16 or 17, wherein said surface comprises silicon.

24. The method of claim 16 or 17, wherein said surface is selected from the group consisting of: flow cells, sequencing flow cells, flow channels, microfluidic channels, capillary tubes, piezoelectric surfaces, wells, microwells, microwell arrays, microarrays, chips, wafers, non-magnetic beads, magnetic beads, ferromagnetic beads, paramagnetic beads, superparamagnetic beads, and polymer gels.

25. The method of claim 16 or 17, wherein said initiator species comprises an organosilane.

26. The method of claim 16 or 17, wherein said initiator species comprises the molecule shown in FIG. 1.

27. The method of claim 16 or 17, wherein said surface initiated polymerization comprises atom-transfer radical polymerization (ATRP).

28. The method of claim 16 or 17, wherein said surface initiated polymerization comprises reversible addition fragmentation chain-transfer (RAFT).

29. The method of claim 1, wherein said biomolecules comprise 5′ acrydite modified oligonucleotides.

30. The method of claim 1, wherein said biomolecules comprise antibodies.

31. The method of claim 1, wherein said biomolecules comprise peptides.

32. The method of claim 1, wherein said biomolecules comprise aptamers.

33. The method of claim 1, wherein the coupling of the biomolecules comprises incorporation of acrydite-modified biomolecules during polymerization.

34. The method of claim 1, wherein the coupling of the biomolecules comprises reaction at bromoacetyl sites.

35. The method of claim 1, wherein the coupling of the biomolecules comprises reaction at azide sites.

36. The method of claim 1, wherein the coupling of the biomolecules comprises azide-alkyne Huisgen cycloaddition.

37. A composition, comprising:
(a) a surface;
(b) a polymer coating covalently bound to said surface, formed by surface-initiated polymerization, wherein the polymer coating comprises 2 or more different types of acrylamide monomers; and
(c) a biomolecule coupled to said polymer coating.

38. A composition, comprising:
(a) a surface;
(b) a polymer coating covalently bound to said surface, formed by surface-initiated polymerization; and
(c) at least two different biomolecules coupled to said polymer coating.

39. The composition of claim 37, wherein said biomolecule comprises an oligonucleotide.

40. The composition of claim 39, wherein said oligonucleotide is coupled to the polymer at its 5′ end.

41. The composition of claim 39, wherein said oligonucleotide is coupled to the polymer at its 3′ end.

42. The composition of claim 37, wherein said biomolecule comprises an antibody.

43. The composition of claim 37, wherein said biomolecule comprises an aptamer.

44. The composition of claim 38, wherein said at least two different biomolecules comprise oligonucleotides.

45. The composition of claim 44, wherein said oligonucleotides are coupled to the polymer coating at their 5′ ends.

46. The composition of claim 44, wherein said oligonucleotides are coupled to the polymer coating at their 3′ ends.

47. The composition of claim 38, wherein said at least two different biomolecules comprise antibodies.

48. The composition of claim 38, wherein said at least two different biomolecules comprise aptamers.

49. The composition of claim 37 or 38, wherein said surface comprises glass.

50. The composition of claim 37 or 38, wherein said surface comprises silicon.

51. The composition of claim 37 or 38, wherein said polymer coating comprises polyacrylamide.

52. The composition of claim 37 or 38, wherein said polymer coating comprises PMMA.

53. The composition of claim 37 or 38, wherein said polymer coating comprises polystyrene.

54. The composition of claim 37 or 38, wherein said surface-initiated polymerization comprises atom-transfer radical polymerization (ATRP).

55. The composition of claim 37 or 38, wherein said surface-initiated polymerization comprises reversible addition fragmentation chain-transfer (RAFT).
